(19) United States
(12) Patent Application Publication
(10) Pub. No.: US 2004/0170699 A1
(43) Pub. Date: Sep. 2, 2004

(54) Hydroxyapatite dispersions comprising an amino acid as stabilizing agent and method for preparing same

(76) Inventors: Jean-Yves Chane-Ching, Eaubonne (FR); Claude Magnier, Paris (FR); Emmanuel Vignaud, Chatenay-Malabry (FR)

Correspondence Address: BURNS DOANE SWECKER & MATHIS L L P, POST OFFICE BOX 1404, ALEXANDRIA, VA, 22313-1404, US

(21) Appl. No.: 10/474,024

(22) PCT Filed: Apr. 25, 2002
(86) PCT No.: PCT/FR02/01440

(30) Foreign Application Priority Data
Apr. 27, 2001 (FR) 01/05747

(51) Int. Cl. A61K 33/42

(52) U.S. Cl. 424/602

(57) ABSTRACT

The invention concerns a stable aqueous colloidal dispersion of colloids with apatite structure, having a pH ranging between 5 and 10, consisting of oblong colloids with an average number length ranging between 20 and 250 nm and an equivalent aspect ratio (number average length/equivalent diameter ratio) ranging between 1 and 300 or in spherical shape having a diameter ranging between 10 and 100 nm, and comprising one or several amino acids optionally in ionized form, as stabilising agents for said colloids with apatite structure corresponding to formula (I): Ca₁₀₋ₓ(HPO₄)ₓ(PO₄)₆₋ₓ(J)₂₋ₓ, wherein: x and J are such as defined in claim 1.

The present invention relates to stable aqueous colloidal dispersions of colloids possessing an apatite structure in which the colloids, of oblong or spherical shape, exhibit nanometric dimensions.

These dispersions are stabilized by stabilizing agents of amino acid type, optionally in the ionized form, in interaction with the surface of the colloids.

The colloids of oblong shape are in the more or less aggregated form and exhibit a (number-)average length generally of between 20 and 250 nm and an equivalent aspect ratio (ratio of the (number-)average length to the equivalent diameter) of between 1 and 300.

The colloids of spherical shape exhibit a diameter of between 10 and 100 nm, preferably between 10 and 60 nm, for example between 10 and 40 nm.

The term “aqueous colloidal dispersion” is generally understood to mean a system composed of a continuous aqueous phase in which fine solid- particles of colloidal dimensions are dispersed, said fine particles defining colloids at the surface of which molecules Of a stabilizing agent or various ionic entities present in the continuous aqueous phase can be bonded or adsorbed.

The term “colloids possessing an apatite structure” is understood to mean, according to the invention, colloids of general formula:

Ca₁₀₋ₓ(HPO₄)ₓ(PO₄)₆₋ₓ(J)₂₋ₓ (I)

in which:

x is selected from 0, 1 or 2;

J is selected from OH⁻, F⁻, CO₃²⁻ and/or Cl⁻; and in which some phosphate ions (PO₄³⁻) or hydrogen-phosphate ions (HPO₄²⁻) can be replaced by carbonate ions (CO₃²⁻);

and in which some Ca²⁺ cations can be replaced by Mⁿ⁺ metal cations of alkali metals, alkaline earth metals or lanthamide metals where n represents. 1, 2 or 3,

it being understood that the molar ratio of the Mⁿ⁺ cation, when it is present, to Ca²⁺ varies between 0.01:0.99 and 0.25:0.75, and that the substitution of HPO₄²⁻ ions or of PO₄³⁻ ions by CO₃²⁻ ions, the incorporation of CO₃²⁻ ions as J and the substitution of Ca²⁺ cations by metal cations is carried out so as to satisfy the electronic balance, in particular with creation of gaps.

In a particularly preferred way, when Ca²⁺ is replaced by an alkali metal cation, the latter is Na⁺. When Ca²⁺ is replaced by an alkaline earth metal cation, the latter is Sr²⁺.

When Ca²⁺ is replaced by a lanthamide cation, the latter is preferably Eu³⁺, Eu²⁺, Dy³⁺ or Tb³⁺.

More generally, the term “lanthanide” is understood to mean the elements from the group consisting of yttrium and of the elements of the Periodic Table with an atomic number between 57 and 71, inclusive.

The Periodic Table of the Elements to which reference is made in the present description is that published in the Supplement to the Bulletin de la Société Chimique de France, No. 1 (January 1966).

When x=0, the colloids are hydroxyapatite colloids. When x=1, the colloids are apatitic tricalcium phosphate crystals and, when x=2, the colloids are octocalcium phosphate crystals.

The expression “colloids possessing an apatite structure” also encompasses colloids obtained by hydrolysis of the colloids of formula I above.

In the case of octocalcium phosphate, a colloid of formula Ca₈(HPO₄)_{2.5}(PO₄)_{3.5}OH_{0.5} is obtained after hydrolysis.

In the above formula, it is preferable for no Ca²⁺ cation to be replaced by an Mⁿ⁺ metal cation. However, when some Ca²⁺ cations are actually replaced by Mⁿ⁺ metal cations, then it is preferable for the Mⁿ⁺/Ca²⁺ molar ratio to be between 0.02/0.98 and 0.15/0.85.

Colloids possessing an apatite structure are generally obtained by bringing into contact, in aqueous solution, a source of Ca²⁺ and a source of PO₄³⁻ in an appropriate pH range.

Conventionally, colloids possessing an apatite structure, the growth of which is difficult to control and limit, are obtained.

The kinetics of formation of the particles are often very high, so that it is difficult to halt the inorganic polycondensation at the stage of nanometric particles. Thus, in fine, excessively large particles exhibiting a strong tendency to separate by settling are generally obtained.

The invention provides, according to a first of its aspects, a process which makes it possible to control the growth of colloids possessing an apatite structure and which results in stable colloidal dispersions composed of colloids of nanometric dimensions.

According to another of its aspects, the invention relates to stable aqueous colloidal dispersions of colloids possessing an apatite structure formed of relatively fine colloids, of oblong shape, with a (number-)average length of between 20 and 250 nm and with an equivalent aspect ratio (ratio of the (number-)average length to the equivalent diameter) of between 1 and 300, or else of spherical shape, exhibiting a diameter of between 10 and 100 nm. These dispersions are generally formed of weakly aggregated colloids. In the case of colloids which are small in size and which are weakly aggregated, the dispersions of the invention are transparent to the naked eye.

The term “weakly aggregated colloids” is understood to mean a percentage by number of completely separate objects of greater than 80%, preferably of greater than 90%, advantageously of greater than 95%.

More specifically, the invention relates to a stable aqueous colloidal dispersion of colloids having an apatite structure, exhibiting a pH of between 5 and 10, composed of colloids of oblong shape with a (number-)average length of between 20 and 250 nm and with an equivalent aspect ratio (ratio of the (number-)average length to the equivalent diameter) of between 1 and 300, or of spherical shape exhibiting a diameter of between 10 and 100 nm and comprising one or more amino acids, optionally in the ionized form, as stabilizing agent; wherein said colloids having an apatite structure have the formula:

Ca₁₀₋ₓ(HPO₄)ₓ(PO₄)₆₋ₓ(J)₂₋ₓ (IV)

in which:

x is selected from 0, 1 or 2;

J is selected from OH⁻, F⁻, CO₃²⁻ or Cl⁻;

and in which some phosphate ions (PO₄³⁻) or hydrogen-phosphate ions (HPO₄²⁻) can be replaced by carbonate ions (CO₃²⁻);

and in which some Ca²⁺ can be replaced by Mⁿ⁺ metal cations of alkali metals, alkaline earth metals or lanthanide metals where n represents 1, 2 or 3, it being understood that the molar ratio of the M^{N+} cation, when it is present, to Ca²⁺ varies between 0.01:0.99 and 0.25:0.75, and that the substitution of HPO₄²⁻ ions or of PO₄³⁻ ions by CO₃²⁻ ions, the incorporation of CO₃²⁻ ions as J and the substitution of Ca²⁺ cations by metal cations is carried out so as to satisfy the electronic balance, in particular with creation of gaps.

In the context of the invention, the term “colloids of oblong shape” is understood to mean colloids of parallelepipedal shape (for example in the shape of a rod) or of acicular shape.

In the case of colloids of parallelepipedal shape, the equivalent diameter is the diameter which the corresponding colloid of acicular shape with the same average volume and the same average length would have.

The equivalent diameter assigned to the cross section of the acicular colloid corresponds to the diameter of an average cross section.

The colloids of oblong shape are formed of colloids possessing a weakly aggregated apatite structure. Generally, the colloids of oblong shape exhibit a (number-)average length of between 20 and 250 nm and an equivalent diameter of between 0.5 and 20 nm.

The following are more particularly distinguished: colloids in the shape of acicular fibers, the average length of which generally varies between 20 and 250 nm and the equivalent diameter of which is between 0.5 and 5 nm; and colloids in the shape of rods, the average length of which generally varies between 20 and 250 nm and the equivalent diameter of which is between 5 and 20 nm.

The spherical colloids have a diameter generally. of between 10 and 100 nm, preferably between 10 and 60 nm, better still of between 10 and 40 nm.

The dispersions of the invention are either uniformly formed of colloids of oblong shape, or uniformly formed of spherical colloids, or alternatively formed of a mixture of colloids of oblong shape and of spherical shape.

The colloids possessing apatite structures synthesized are preferably colloids of formula (I) in which x=0, better still colloids of formula: Ca₁₀(PO₄)₆(OH)₂.

More specifically, it is preferable, in the formula (I), for J to represent OH⁻ or/and F⁻. It is not necessary for all the OH⁻ ions to be replaced by F⁻ ions but only a portion of the OH⁻ ions may be replaced by F⁻ ions.

Likewise, when J is selected from OH⁻, F⁻, CO₃²⁻ and Cl⁻, it is not necessary. for all the J groups to be identical to one another.

The stabilization of the colloidal dispersion is obtained by the action of a stabilizing agent. The stabilizing agent contributes not only to stabilizing the dispersion but also to controlling the growth of the colloids possessing an apatite structure during the preparation of the aqueous dispersion.

In the context of the invention, the stabilizing agent is a natural or synthetic amino acid, optionally in the ionized form, or a mixture of these compounds.

α-Amino acids comprise a carbon atom carrying an amino group, a carboxyl group, a hydrogen atom and a side group which can be a hydrogen atom (case of glycine) or any other monovalent organic group.

The side groups can in particular be alkyl groups (case of alanine, valine, leucine, isoleucine and proline), substituted alkyl groups (case of threonine, serine, methionine, cysteine, asparagine, aspartic acid, glutamic acid, glutamine, arginine and lysine), arylalkyl groups (case of phenylalanine and tryptophan), substituted arylalkyl groups (case of tyrosine) or heteroalkyl groups (case of histidine).

These α-amino acids are listed in particular in Harper et al. (1977), Review of Physiological Chemistry, 16^{th} edition, Lange Medical Publications, pages 21-24.

According to the invention, the expression “amino acid” also comprises β-, γ-, δ- and ω-amino acids.

The term “synthetic α-amino acid” is understood to mean an α-amino acid which is not incorporated in a protein under the control of mRNA, such as, for example, a fluorinated α-amino acid, such as fluoroalanine, trimethylsilylalanine or an α-amino acid such as:

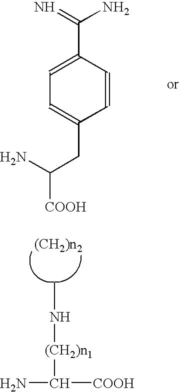

where n₁ is an integer from 1 to 6 and n₂ is an integer from 1 to 12.

Synthetic amino acids are furthermore described in Williams (editor), Synthesis of Optically Active α-Amino Acids, Pergamon Press (1989); Evans et al., J. Amer. Chem. Soc., 112, 4011-4030 (1990); (Pu et al., J. Amer. Chem. Soc., 56, 1280-1283 (1991); or Williams et al., J. Amer. Chem. Soc., 113, 9276-9286 (1991).

The amino acids which can be used as stabilizing agents 10 are in their L form or in their D form or alternatively in the form of a racemic mixture.

More generally, a preferred α-amino acid group is composed of the compounds of formula:

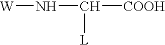

in which

L represents an alkyl group optionally interrupted by an oxygen atom and/or a sulfur atom and/or a nitrogen atom, said nitrogen atom carrying a hydrogen atom or an alkyl, aryl, arylalkyl, alkylaryl, heteroaryl or heteroarylalkyl radical and said alkyl group optionally being substituted by one or more radicals selected from —OH, —NH₂, guanidino, carboxyl, carbamoyl, thiol, aryl (itself optionally substituted by one or more radicals T, which are identical or different, as defined below) or heteroaryl (itself optionally substituted by one or more radicals T, which are identical or different, as defined below);

W represents a hydrogen atom or else L and W together represent an optionally substituted alkylene chain;

T represents hydroxyl, amino, guanidino, carboxyl, thiol, alkylthio, alkylamino, carbamoyl, dialkylamino, aryl, arylalkyl, alkylaryl, heteroaryl, alkylheteroaryl or heteroarylalkyl.

The term “alkylene” is understood to mean a linear or branched aliphatic hydrocarbonaceous chain.

The substituents of the alkylene chain are selected from the T groups defined above.

The term “alkyl” is generally understood to mean a linear or branched aliphatic hydrocarbonaceous chain comprising from 1 to 18 carbon atoms, preferably from 1 to 10 carbon atoms and in particular from 1 to 6 carbon atoms.

Examples of alkyl radicals are the methyl, ethyl, propyl, isopropyl, butyl, isobutyl, t-butyl, pentyl, isopentyl, neopentyl, 2-methylbutyl, 1-ethylpropyl, hexyl, isohexyl, neohexyl, 1-methylpentyl, 3-methylpentyl, 1,1-dimethylbutyl, 1,3-dimethylbutyl, 2-ethylbutyl, 1-methyl-1-ethylpropyl, heptyl, 1-methylhexyl, 1-propylbutyl, 4,4-dimethylpentyl, octyl, 1-methylheptyl, 2-ethylhexyl, 5,5-dimethylhexyl, nonyl, decyl, 1-methylnonyl, 3,7-dimethyloctyl and 7,7-dimethyloctyl radicals.

More particularly, alkyl represents methyl, ethyl, propyl, isopropyl, butyl, t-butyl, pentyl, isopentyl, neopentyl, 2-methylbutyl, 1-ethylpropyl, hexyl, isohexyl, neohexyl, 1-methylpentyl, 3-methylpentyl, 1,1-dimethylbutyl, 1,3-dimethylbutyl, 2-ethylbutyl and 1-methyl-1-ethylpropyl.

Aryl generally denotes an aromatic carbocyclic radical comprising from 6 to 18 carbon atoms, preferably from 6 to 10 carbon atoms.

Aryl is mono- or polycyclic and preferably mono-, bi- or tricyclic. When the carbocyclic radical comprises more than one ring, the rings can be fused in pairs or attached in pairs via σ bonds.

Aryl group examples are phenyl, anthryl, naphthyl or phenanthryl.

The term “heteroaryl” is generally understood to mean heterocyclic radicals comprising one or more hetero-atoms selected from O, S and N.

Heteroaryl radicals encompass mono- and polycyclic radicals; preferably mono-, bi- or tricyclic radicals.

In the case of polycyclic radicals, it should be understood that the latter are composed of monocycles fused in pairs (for example ortho-fused or peri-fused), that is to say including at least two carbon atoms in common. Preferably, each monocycle comprises from 3 to 8 members, better still from 5 to 7.

Preferably, each of the monocycles constituting the heterocycle comprises from 1 to 4 heteroatoms, better still from 1 to 3 heteroatoms.

The following in particular are distinguished:

-5- to 7-membered monocyclic heterocycles, such as, for example, heteroaryls selected from pyridine, furan, thiophene, pyrrole, pyrazole, imidazole, thiazole, isoxazole, isothiazole, furazan, pyridazine, pyrimidine, pyrazine, thiazines, oxazole, pyrazole, oxadiazole, triazole and thiadiazole;

bicyclic heteroaryls in which each monocycle comprises from 5 to 7 ring members, such as, for example, selected from indolizine, indole, isoindole, benzofuran, benzothiophene, indazole, benzimidazole, benzothiazole, benzofurazan, benzothiofurazan, purine, quinoline, isoquinoline, cinnoline, phthalazine, quinazoline, quinoxaline, naphthyridines, pyrazolotriazine (such as pyrazolo[1,3,4]triazine), pyrazolopyrimidine and pteridine; and

tricyclic heteroaryls in which each monocycle comprises from 5 to 7 ring members, such as, for example, acridine, phenazine or carbazole.

The expression “optionally interrupted by O and/or S and/or N” means that any carbon atom of the hydrocarbonaceous chain can be replaced by an oxygen and/or sulfur and/or nitrogen atom, it not being possible for this carbon atom to be situated at one of the ends of said hydrocarbonaceous chain. The hydrocarbonaceous chain, which can be an alkyl chain, can comprise several oxygen and/or sulfur and/or nitrogen atoms, the heteroatoms preferably being separated from one another by at least one carbon atom, better still by at least two carbon atoms.

When the alkylene chain is interrupted by a nitrogen atom, it is preferable for the latter to carry a hydrogen atom or an alkyl group.

In a particularly advantageous way, the stabilizing agent used is selected from lysine, glycine, asparagine, creatine, arginine, aspartic acid, glutamic acid, serine, alanine, valine, leucine, their salts with acids or bases, and their mixtures.

In an even more preferred way, the stabilizing agent is selected from lysine, creatine, glycine, alanine, asparagine, serine, their salts with acids or bases, and their mixtures.

The stabilizing agent can also be the salt of an amino acid with a base or an acid, preferably with an inorganic acid or base.

Mention may be made, as example of inorganic acid, of nitric, phosphoric, phosphinic, phosphonic, hydrochloric, sulfonic and sulfuric acids.

Mention may be made, as example of inorganic base, of bases of alkali metal hydroxide, alkaline earth metal hydroxide and ammonium hydroxide type.

The stabilizing agent can be composed of one or more amino acids, optionally in the ionized form.

The stabilizing agent is generally either present in the free form in the continuous medium of the colloidal dispersion, or adsorbed at or bonded to the surface of the colloids, or in interaction with Ca²⁺ ions present in the continuous phase of the dispersion.

The colloidal phase predominantly possesses an apatite structure as defined above. Advantageously, the apatite structure represent more than 50% by weight of the colloidal phase, preferably more than 75% by weight, better still more than 80%, for example more than 85% by weight.

The colloidal phase can additionally comprise other structures, such as Ca(H₂PO₄)₂; CaHPO₄; CaHPO₄.2H₂O, or other amorphous phase based on calcium and on PO₄³⁻, HPO₄²⁻ or H₂PO₄⁻ and OH⁻.

According to a preferred embodiment of the invention, the molar ratio of the total calcium present in the colloids to the total phosphorus present in the colloids varies between 1.3 and 1.7, better still between 1.5 and 1.67. With regard to the molar ratio of the total stabilizing agent present in the colloids or at the surface of the colloids to the total calcium present in the colloids or at the surface of the colloids, it varies between 0.001 and 1.0, preferably between 0.01 and 0.5, advantageously between 0.01 and 0.1.

In a particularly preferred way, the colloidal phase comprises from 60 to 100% of the total calcium, preferably from 80 to 100%, for example from 90 to 100%, better still from 95 to 100%.

Advantageously, the colloidal phase comprises from 80 to 100% of the total phosphorus (total PO₄₃, HPO₄² and H₂PO₄⁻ions), preferably from 90 to 100%, better still from 95 to 100% by weight.

The concentration of calcium in the dispersion can be easily adjusted, according to the invention, by removing a portion of the continuous aqueous phase.

The removal of a portion of the aqueous phase can be carried out by ultrafiltration.

However, preferably, the colloidal dispersion of the invention exhibits a concentration of calcium in the form of colloids possessing an apatite structure of greater than 0.25 M, preferably of greater than 0.5 M, advantageously of greater than 1M, it being possible for this concentration to reach 5M.

According to a preferred form of the invention, the pH of the colloidal dispersion of the invention varies between 5 and 9.5, better still between 6.5 and 8.5, for example between 6 and 8.

According to another of its aspects, the invention relates to a process for the preparation of a stable aqueous colloidal dispersion comprising the stages consisting in:

a) bringing into contact, in aqueous solution, a source of Ca²⁺ cations and a source of PO₄³⁻ anions and an amino acid as stabilizing agent of one of salts with an acid or a base, at a pH of between 5 and 10, the respective amounts of the source of Ca²⁺ and of the source of PO₄³⁻ anions being such that the Ca²⁺/P molar ratio varies between 1 and 3.5, preferably between 2 and 3.2, the amount of stabilizing agent being such that the stabilizing agent/Ca molar ratio varies between 0.3 and 2.5, preferably between 0.9 and 2;

b) leaving the solution thus obtained to mature at a temperature of between 15 and 150° C. until a colloidal dispersion is obtained.

The term “source of Ca²⁺ cations” is understood to mean a compound capable of releasing Ca²⁺ ions in aqueous solution.

The term “source of PO₄³⁻ anions” is understood to mean a compound capable of releasing PO₄³⁻ ions in aqueous solution.

Examples of source of Ca²⁺ cations are calcium hydroxide, calcium oxides or water-soluble calcium salts.

Examples of calcium salts are salts having, as anion, PF₆⁻, PCl₆⁻, BF₄⁻, BCl₄⁻, SbF₆⁻, BPh₄⁻, ClO₄⁻, CF₃SO₃⁻ and more generally carboxylates derived from C₂-C₁₀ alkylcarboxylic acids and in particular the acetate. Other salts are calcium halides, calcium hydrogencarbonate and calcium nitrate. Among these salts, those which can be used in the context of the invention are those exhibiting a sufficient solubility in water to provide the desired concentration of Ca²⁺ in the aqueous phase.

In a particularly preferred way, the source of Ca²⁺ cations is selected from calcium hydroxide, calcium oxides, calcium halides, calcium nitrate and calcium hydrogencarbonate.

By way of example, the source of PO₄³⁻ anions is the salt of a PO₄³⁻ anion, the salt of an HPO₄²⁻ anion or the salt of an H₂PO₄⁻ anion, such as an ammonium salt or an alkali metal salt or a mixture of these salts.

Other sources of PO₄³⁻ are the salts of the anions of oligomeric phosphate type, such as the salts of polyphosphates (or catena-polyphosphate) of general formula:

[O(PO₃)ₙ]⁽ⁿ⁺²⁾⁻

in which n varies from 2 to 10 (and in particular the salts of tripolyphosphate type), or the salts of the trimetaphosphate anion (PO₃)₃³⁻ or the salts of the pyrophosphate anion (P₂O₇)⁴⁻.

The use of the acid H₃PO₄ can also be envisaged as source of PO₄³⁻ anions.

Advantageously, when a calcium oxide or calcium hydroxide is used as source of Ca²⁺, it is desirable to select phosphoric acid as source of PO₄³⁻.

These two sources have to be brought together under highly specific pH conditions in order to result in the formation of colloids possessing the desired apatite structure: generally a pH of between 5 and 10, preferably between 5 and 9.5, better still between 7 and 9.2, is highly suitable.

After bringing the two sources together in an aqueous medium, it may therefore prove to be necessary to adjust the pH of the aqueous medium by addition to this medium of an acid or of a base, preferably an inorganic acid or base.

The bases and acids which can be used are those generally used in the art.

Mention may be made, as bases which can be used, of NH₄OH, KOH, NaOH, NaHCO₃, Na₂CO₃, KHCO₃ and K₂CO₃.

Use will preferably be made of NH₄OH or NaOH.

Examples of acids which can be used are in particular HCl, H₂SO₄, H₃PO₄ or HNO₃. Use will preferably be made of HNO₃ or HCl.

A buffer operating within the desired range can be used to adjust the pH. Use is preferably made of a buffer providing a pH of 6.5 to 9.

Mention may be made, as particularly preferred example, of a buffer composed of an aqueous solution of potassium dihydrogenphosphate (0.025M) and of sodium hydrogenphosphate (0.025M), which provides a pH of 6.86 at 25° C.

The sources can be brought into contact in an eous medium in any way.

Preferably, it is recommended to prepare, in a first step, an aqueous solution of the source of Ca²⁺, on the one hand, and an aqueous solution of the source of PO₄³⁻, on the other hand. The relative proportions of the compounds used respectively as source of Ca²⁺ and of PO₄³⁻ are calculated so that the Ca/P molar ratio is between 1 and 3.5, preferably between 2 and 3.2.

The Ca/P molar ratio takes into account all the Ca²⁺ cations introduced and all the phosphorus introduced into the solution, whether the phosphorus is in the H₃PO₄, H₂PO₄³⁻, HPO₄²⁻ or PO₄³⁻ form.

The stabilizing agent is then added, either to the aqueous Ca²⁺ solution or to the aqueous PO₄³⁻ solution or to both aqueous solutions, in which case the respective proportion of stabilizing agent added to each solution can take any value.

Preferably, the stabilizing agent is added to the aqueous Ca²⁺ solution.

The amount of stabilizing agent to be added in total is defined so that the stabilizing agent/Ca molar ratio varies between 0.3 and 2.5, preferably between 0.9 and 2.

The amount of stabilizing agent used changes the dimensions of the colloids finally obtained. It is in particular by controlling this parameter that it is possible to result in the production of transparent aqueous dispersions.

A molar ratio of the stabilizing agent to the Ca²⁺ of between 1.0 and 2 results in particular in transparent dispersions.

The following stage consists in mixing the two aqueous solutions, this mixing being carried out conventionally with stirring.

Preferably, a preliminary adjustment of the pH of the two solutions is carried out before mixing. This pH can be adjusted to between 5 and 11, preferably between 7 and 9.5.

Advantageously, after mixing, the concentration of Ca²⁺ cations in the solution is between 0.2 and 2M, preferably between 0.2 and 1M; the concentration of total PO₄³⁻, HPO₄²⁻ and H₂PO₄⁻ ions varies between 0.1M and 1M, preferably between 0.1 and 0.5M; and the concentration of stabilizing agent is between 0.1M and 3M.

After mixing, it may prove to be necessary to again adjust the pH of the solution under the conditions described above.

The mixing can be carried out either by addition of the solution of the source of Ca²⁺, optionally comprising the stabilizing agent, to the solution of the source of PO₄³⁻, optionally comprising the stabilizing agent, or vice versa.

This addition can be carried out instantaneously or gradually and at a constant flow rate. In the case of an addition at a constant flow rate, this addition can be carried out over a period of 15 min to 6 hours, preferably of 15 min to 4 hours, advantageously 15 min to 1 hour.

Preferably, the solution of the source of PO₄³⁻ will be gradually added to the solution of the source of Ca²⁺ comprising the stabilizing agent.

For the purpose of preparing colloids in which some of the calcium cations are replaced by metal cations, it is necessary to add one or more sources of said metal cations to the reaction medium. Appropriate sources are composed of hydroxides of these metals or salts of these metals, such as the halides or nitrates.

In the case where the metal cation is the cation of a lanthanide, it is preferable to add a salt of said lanthanide to the reaction solution, such as a chloride or a nitrate. This salt will be added, for example, to the solution of the source of calcium before it is mixed with the source of PO₄³⁻.

The source of Ca²⁺ and the source of PO₄³⁻ are generally brought into contact at ambient temperature, for example between 15 and 30° C.

Stage b) of the process of the invention is a maturing stage during which the mixture of the two solutions is left standing or stirring, the time necessary to observe the formation of colloids.

In stage b), the colloidal dispersion resulting from stage a), which is a milky dispersion, changes to a colloidal dispersion which is stable with regard to separation by settling.

This maturing stage can be carried out at ambient temperature (15-30° C.) or at a higher temperature, namely at 180° C. Thus, generally, the temperature is set at this stage between 15 and 180° C., better still between 40 and 160° C.

According to a preferred embodiment of the invention, the maturing is carried out in a closed chamber at a temperature of less than 100° C. and in an autoclave at a temperature of greater than 100° C.

When the maturing stage is carried out at a temperature of less than 100° C. in a closed chamber, the colloids obtained preferably have an anisotropic morphology.

Conversely, when the maturing is carried out in an autoclave at a temperature of greater than 100° C., a mixture of colloids possessing anisotropic morphology and of colloids possessing isotropic morphology is obtained.

The maturing stage is preferably carried out in a closed chamber.

The dispersion, conditioned in a closed chamber, can be placed directly in an oven brought beforehand to the set temperature or can be subjected to a temperature gradient up to the set temperature, the rate of temperature rise preferably varying between 0.1° C./min and 10° C./min.

According to another embodiment of the invention, the maturing is carried out at various temperatures.

Preferably, a first phase of the maturing is carried out at a first temperature of between 20 and 180° C. and a second maturing phase is carried out at a second temperature, said second temperature also being between 20 and 180° C. Advantageously, the second temperature is greater than said first temperature.

The maturing time varies according to the operating conditions and more particularly the temperature. The maturing time usually varies between 15 min and 24 hours.

The continuous phase of the colloidal dispersion can comprise various entities, such as NH₄⁺, Na⁺, K⁺, Cl⁻, NO₃⁻ and SO₄². These ions originate either from the sources of calcium and of PO₄³⁻ or from the inorganic acids and bases used for the pH adjustments.

The continuous phase of the colloidal dispersion can also comprise stabilizing agents, in neutral form or in ionized form, not in interaction with the surface of the colloids, that is to say completely free, or in interaction with Ca²⁺ ions present in the continuous phase of the dispersion.

It is difficult to avoid the presence of various calcium or phosphorus entities and the presence of the stabilizing agent in the continuous aqueous phase or beside the colloids with apatite structure, so that it may be necessary to carry out a purification, for example by washing the dispersion.

This washing operation can be carried out in a way conventional per se, by ultrafiltration or dialysis.

Ultrafiltration can be carried out in particular under air or under an atmosphere of air and of nitrogen or under nitrogen. It is preferably carried out with water having a pH adjusted to the pH of the dispersion and is, for example, carried out using 3 kD or 15 kD membranes.

If appropriate, the dispersion can then also be concentrated by removing a portion of the continuous phase. The most appropriate technique for doing this is the ultrafiltration technique.

If appropriate, it may be of use to adjust the pH of the final dispersion by addition of an acid or of a base, preferably an inorganic acid or base, such as those defined above. The pH of the final dispersion will advantageously be adjusted to between 6 and 8.

The size of the colloids can be determined by photometric counting from an HRTEM (High Resolution Transmission Electron Microscopy) analysis. The structure of the colloids and in particular their greater or lesser degree of aggregation can be determined by cryo-transmission electron microscopy by following the Dubochet method.

The (number-)average length of the colloids of oblong shape varies between 20 and 250 nm and their equivalent aspect ratio (ratio of the (number-)average length to the equivalent diameter) varies between 1 and 300.

As regards the colloids of spherical shape, their diameter varies between −10 and 100 nm.

The colloidal dispersions of the invention can be used in many applications, as they are or after isolation of the colloids possessing an apatite structure to form porous materials.

The colloidal dispersions of the invention can also be used after preparation of an emulsion by addition of an oily phase.

Applicational examples of the colloidal dispersions or of the porous materials are the separation and purification of proteins, use in prostheses and use in prolonged release systems.

The colloids can be isolated in a way known per se: simple evaporation at ambient temperature, evaporation under vacuum, evaporation at a temperature of greater than 100° C., by ultracentrifuging or, preferably, drying/atomization.

Drying/atomization can be described as an atomization of the colloidal dispersion using a nozzle in a temperature chamber. Examples of industrial dryers/atomizers are the dryers/atomizers of the Niro or Buchi type. Preferably, redispersible colloids are obtained for outlet temperatures of less than 150° C.

Thus, according to another of its aspects, the invention relates to water-redispersible colloids possessing an apatite structure which can be obtained by carrying out the stages consisting in:

a) preparing a colloidal dispersion by employing the process described above;

b) isolating in a way known per se, and preferably by centrifuging, the colloids from the colloidal dispersion resulting from stage a).

In the pharmaceutical field, the hydroxyapatite colloids obtained can be used in the treatment of osteoporosis, cramp, colitis, bone fractures or insomnia and in dental hygiene.

The hydroxyapatite colloids can be used in the preparation of hydroxyapatite films, of absorbent materials with a high specific surface and with a high pore volume, of encapsulation materials and of catalytic materials, but also in the field of luminescence.

The colloids of the aqueous dispersions of the invention can be isolated simply by ultracentrifuging. These colloids can exhibit, bonded to or adsorbed at their surface, a certain amount of stabilizing agent. The amount of stabilizing agent present can be determined by chemical quantitative. determination.

The percentage by mass of Ca in the colloids is determined from the colloids isolated by centrifuging and dried at ambient temperature for 7 days, in the following way.

The dried colloids are dissolved by HNO₃/HF/H₂O₂ using microwave radiation. The Ca is then quantitatively determined by inductively coupled plasma/atomic emission spectroscopy ICP/AES on a Jobin Yvon Ultima device. The principle is that the atoms are excited in an argon plasma, with emission of photons of different wavelengths. A grating spectrometer makes possible separation of the wavelengths and detection is carried out using a photomultiplier. Likewise, a percentage by mass of carbon on the colloids recovered by ultracentrifuging and dried at ambient temperature ror 7 days is determined by analysis with a Leco CS-044. The product is oxidized in the presence of catalyst in an induction furnace while flushing with oxygen. The CO₂ peaks are detected and integrated by infrared spectrometry.

The determination is thus carried out, from these analyses, of a C/Ca experimental ratio by mass and, by calculation, of the “stabilizing agent/Ca” molar ratio of the colloids.

In a particularly advantageous way, the colloidal dispersion obtained is transparent to the naked eye. Colloidal dispersions transparent to the naked eye are formed of poorly aggregated, well separated colloids. For these transparent dispersions, at least 80% by number of the colloids, preferably at least 90% and advantageously at least 95% by number, are not aggregated. This state of aggregation can be revealed by cryo-transmission electron microscopy, according to the Dubochet method. This method makes it possible to observe, by transmission electron microscopy (TEM), samples kept frozen in their natural medium, which is either water or organic diluents. Freezing is carried out on thin films with a thickness of approximately 50 to 100 nm, either in liquid ethane, for the aqueous samples, or in liquid nitrogen, for the others. The state of dispersion of the particles is well preserved by cryo-TEM and representative of that present in the real medium.

For these dispersions, the length of the colloids preferably varies between 20 and 150 nm, better still it is less than 120 nm, advantageously less than 50 nm, and the diameter of the spheres varies between 10 and 100 nm.

According to another of its aspects, the invention relates to transparent colloidal dispersions formed of colloids of oblong shape with a (number-)average length of 20 to 150 nm or of spherical shape with a diameter of 10 to 100 nm, in which invention at least 80% of the colloids are not aggregated, the molar ratio of the stabilizing agent to the total calcium present in the colloids or at the surface of the colloids varies between 0.001 and 1, preferably between 0.01 and 0.5, the pH of the colloidal dispersion being between 5 and 9.5.

More preferably, the stabilizing agent is selected from alanine and lysine, optionally ionized, or a mixture of these compounds.

It is desirable, so as to obtain such transparent aqueous dispersions, to adjust one or more of the process parameters in the following way:

a) the molar ratio of the stabilizing agent to the calcium is -preferably greater than 0.5:1, better still greater than 1:1;

b) the pH is preferably between 5 and 9.5, better still between 7 and 9.5;

c) the stabilizing agent is composed of one or more amino acids, optionally in the ionized form; it is preferably selected from lysine, alanine and their ionized forms;

d) the source of Ca²⁺ cations, the source of PO₄³⁻ anions and the stabilizing agent are brought into contact by addition of the solution of the source to PO₄³⁻ to the solution of the source of Ca²⁺, which comprises the stabilizing agent, or vice versa.

Consequently, according to another of its aspects, the invention relates to the transparent dispersions which can be obtained by employing the process of the invention in which one or more of the above parameters a) to d) have been selected.

The invention is described more specifically below with reference to specific embodiments of the invention.

Each of the examples below illustrates the preparation of colloidal aqueous dispersions of hydroxyapatite colloids.

The examples below illustrate the preparation of hydroxyapatite colloids.

In the following, M denotes the molecular mass.

### EXAMPLE 1

A solution A is prepared by adding 25.4 ml of 0.98M phosphoric acid, i.e. 25 millimol of phosphorus, to a beaker. The solution is diluted with demineralized water up to a final volume of 60 cm³. The solution is adjusted to pH 9 by addition of 6 cm³ of 10.5M concentrated aqueous ammonia. The solution is made up to 75 cm³ with demineralized water.

A solution B is prepared by adding 12.3 g of Ca(NO₃)₃ (M=164.1 g), i.e. 75 millimol of Ca, and 21.96 g of lysine (M=146 g), i.e. 150 millimol, to a beaker. The mixture is made up to 75 cm³ with demineralized water. It is left stirring until the reactants have completely dissolved. The pH is pH 9.7. The (lysine:Ca) molar ratio is equal to 2.

The solution A is instantaneously added to the solution B at ambient temperature. The Ca/P ratio is equal to 3.

The mixture is left stirring at ambient temperature for 15 min. The pH is pH 9.1.

The mixture is transferred into a closed chamber (Parr bomb, Teflon container) and the mixture is placed in an oven brought beforehand to a temperature of 120° C. The maturing time is 16 hours.

A transparent colloidal dispersion is obtained which has a calcium concentration of 0.5M and which is perfectly stable over time with regard to separation by settling.

Well separated colloids are revealed by transmission electron microscopy, by the cryo-TEM method. The well separated colloids are composed of a population of objects having an anisotropic morphology with an average length of approximately 50 nm and with an equivalent diameter of approximately 10 nm and of a second population possessing a more isotropic, spherical-type, morphology with a diameter of approximately 10 nm.

### EXAMPLE 2

A solution A is prepared by adding 50.8 ml of 0.98M phosphoric acid, i.e. 50 millimol of phosphorus, to a beaker. The solution is diluted with demineralized water up to a final volume of 120 cm³. The solution is adjusted to pH 9 by addition of 12 cm³ of 10.5M concentrated aqueous ammonia. The solution is made up to 150 cm³ with demineralized water.

A solution B is prepared by adding 24.6 g of Ca(NO₃) 2 (M=164.1 g), i.e. 150 millimol of Ca, and 26.6 g of alanine (M=89 g), i.e. 300 millimol, to a beaker. The mixture is made up to 140 cm³ with demineralized water. After the reactants have completely dissolved, the pH is 6.6. The pH is adjusted to pH 9 with 6 cm³ of 10.5M aqueous ammonia and is made up to 150 cm³ with demineralized water. The (alanine:Ca) molar ratio is equal to 2.

The solution A is instantaneously added to the solution B at ambient temperature. The Ca/P ratio is equal to 3. The pH is 8.6. The mixture is adjusted to pH 9 with 6 cm³ of 10.5M concentrated aqueous ammonia.

The mixture is left stirring at ambient temperature for 15 min.

The mixture is transferred into a closed chamber and the mixture is left to mature at ambient temperature of 25° C. After 16 hours, a transparent colloidal dispersion is obtained.

A transparent colloidal dispersion is obtained which has a calcium concentration of 0.5M and which is perfectly stable over time with regard to separation by settling.

Colloids possessing an anisotropic morphology, with an average length of approximately 100 nm and with an equivalent diameter of less than 7 nm, are revealed by transmission electron microscopy, by the cryo-TEM method.

### EXAMPLE 3

A solution A is prepared by adding 50.8 ml of 0.98M phosphoric acid, i.e. 50 millimol of phosphorus, to a beaker. The solution is diluted with demineralized water up to a final volume of 120 cm³. The solution is adjusted to pH 9 by addition of 12 cm³ of 10.5M concentrated aqueous ammonia. The solution is made up to 150 cm³ with demineralized water.

A solution B is prepared by adding 24.6 g of Ca(NO₃)₃ (M=164.1 g), i.e. 150 millimol of Ca, and 22.5 g of glycine (M=75 g), i.e. 300 millimol, to a beaker. The solution is made up to 130 cm³ with demineralized water. The pH is pH 5.2. The solution is adjusted to pH 9 by addition of 12 cm³ of 10.5M aqueous ammonia and is made up to 150 cm³ with demineralized water. The (glycine:Ca) molar ratio is equal to 2.

The solution A is instantaneously added to the solution B at ambient temperature. The Ca/P ratio is equal to 3. The pH is 8.9.

The mixture is left stirring at ambient temperature for 15 min.

The mixture is transferred into a closed chamber and the mixture is placed in an oven brought beforehand to a temperature of 80° C. The maturing time is 16 hours.

A colloidal dispersion is obtained which has a calcium concentration of 0.5M.

200 cm³ of demineralized water are added to 100 cm³ of the dispersion obtained. The dispersion is ultrafiltered over a 3 kD membrane down to a volume of 100 cm³. The operation is repeated a further time.

Colloids composed of a population of objects possessing an anisotropic morphology with an average length of approximately 150 nm and with an equivalent diameter of approximately 10 nm are revealed by transmission electron microscopy carried out on the dispersion, thus washed, by the cryo-TEM method.

### EXAMPLE 4

A solution A is prepared by adding 25.4 ml of 0.98M phosphoric acid, i.e. 25 millimol of phosphorus, to a beaker. The solution is adjusted to pH 9 by addition of 5.5 cm³ of 10.5M concentrated aqueous ammonia. The solution is made up to 75 cm³ with demineralized water.

A solution B is prepared by adding 12.3 g of Ca(NO₃)₃ (M=164.1 g), i.e. 75 millimol of Ca, and 22.5 g of asparagine (M=150 g), i.e. 150 millimol, to a beaker. The solution is made up to 60 cm³ with demineralized water. The pH is pH 3.6. The solution is adjusted to pH 9 by addition of 10 cm³ of 10.5M aqueous ammonia and is made up to 75 cm³ with demineralized water. The (asparagine:Ca) molar ratio is equal to 2.

The solution A is instantaneously added to the solution B at ambient temperature. The Ca/P ratio is equal to 3. The pH is 8.9.

The mixture is left stirring at ambient temperature for 15 min.

The mixture is transferred into a closed chamber and the mixture is placed in an oven brought beforehand to a temperature of 80° C. The maturing time is 16 hours.

A colloidal dispersion is obtained which has a calcium concentration of 0.5M.

200 cm³ of demineralized water are added to 100 cm³ of the dispersion obtained. The dispersion is ultrafiltered over a 3 kD membrane down to a volume of 100 cm³. The operation is repeated a further time.

Aggregated colloids composed of a population of individual objects, the individual objects possessing an anisotropic morphology with an average length of approximately 80 nm, are revealed by transmission electron microscopy carried out on the dispersion, thus washed, by the cryo-TEM method.

### EXAMPLE 5

A solution A is prepared by adding 50.8 ml of 0.98M phosphoric acid, i.e. 50 millimol of phosphorus, to a beaker and diluting with demineralized water up to a final volume of 120 cm³. The solution is adjusted to pH 9 by addition of 24 cm³ of 4M NaOH. The solution is made up to 150 cm³ with demineralized water.

A solution B is prepared by adding 22 g of CaCl₂ (M=147 g), i.e. 150 millimol of Ca, and 26.6 g of alanine (M=89 g), i.e. 300 millimol, to a beaker. The solution is made up to 120 cm³ with demineralized water. The pH is 5.6. The solution is adjusted to pH 9 by addition of 13 cm³ of 4M NaOH and is made up to 150 cm³ with demineralized water. The (alanine:Ca) molar ratio is equal to 2.

The solution A is instantaneously added to the solution B at ambient temperature. The Ca/P ratio is equal to 3. The pH is 8.4. The mixture is adjusted to pH 9 with 7 cm³ of 4M NaOH.

The mixture is left stirring at ambient temperature for 15 min.

The mixture is transferred into a closed chamber and the mixture is left to mature at ambient temperature for 16 hours.

A transparent colloidal dispersion is obtained which is stable over time with regard to separation by settling and which has a calcium concentration of approximately 0.5M.

### EXAMPLE 6

A solution A is prepared by adding 25.4 ml of 0.98M phosphoric acid, i.e. 25 millimol of phosphorus, to a beaker. The solution is adjusted to pH 9 by addition of 6 cm³ of 10.5M concentrated aqueous ammonia. The solution is made up to 37.5 cm³ with demineralized water.

A solution B is prepared by adding 12.3 g of Ca(NO₃)₃ (M=164.1 g), i.e. 75 millimol of Ca, and 21.96 g of lysine (M=146 g), i.e. 150 millimol, to a beaker. The mixture is made up to 37.5 cm³ with demineralized water. After the reactants have completely dissolved, the pH is 9.7. The (lysine:Ca) molar ratio is equal to 2.

The solution A is instantaneously added to the solution B at ambient temperature. The Ca/P ratio is equal to 2.

The mixture is left stirring at ambient temperature for 15 min. The pH is 9.3.

The mixture is transferred into a closed chamber (Parr bomb, Teflon container) and the mixture is placed in an oven brought beforehand to a temperature of 80° C. The maturing time is 16 hours.

A transparent colloidal dispersion is obtained which has a calcium concentration of 1.0M and which is perfectly stable over time with regard to separation by settling.

### EXAMPLE 7

A solution A is prepared by adding 8.5 ml of 0.98M phosphoric acid, i.e. 8.33 millimol of phosphorus, to a beaker and diluting up to a final volume of 20 cm³. The solution is adjusted to pH 9 by addition of aqueous ammonia. The solution is made up to 25 cm³ with demineralized water.

A solution B is prepared by adding 4.1 g of Ca(NO₃)₃ (M=164.1 g), i.e. 25 millimol of Ca, and 2.2 g of lysine (M=146 g), i.e. 15 millimol, to a beaker. The mixture is made up to 25 cm³ with demineralized water and is left stirring until the reactants have completely dissolved. The (lysine:Ca) molar ratio is equal to 0.6.

The solution A is instantaneously added to the solution B at ambient temperature. The Ca/P ratio is equal to 3.

The mixture is left stirring at ambient temperature for 15 min. The pH is 9.1.

The mixture is transferred into a closed chamber (Parr bomb, Teflon container) and the mixture is placed in an oven brought beforehand to a temperature of 80° C. The maturing time is 16 hours.

A transparent colloidal dispersion is obtained which has a calcium concentration of 0.5M and which is perfectly stable over time with regard to separation by settling.

Well separated colloids are revealed by transmission electron microscopy, by the cryo-TEM method. The well separated colloids are composed of a population of objects possessing an anisotropic morphology with an average length of approximately 80 nm and with an equivalent diameter of approximately 10 nm.

1. A stable aqueous colloidal dispersion of colloids possessing an apatite structure, exhibiting a pH of between 5 and 10, formed of colloids of oblong shape with a (number-)average length of between 20 and 250 nm and with an equivalent aspect ratio (ratio of the (number-)average length to the equivalent diameter) of between 1 and 300, or of spherical shape exhibiting a diameter of between 10 and 100 nm and comprising one or more amino acids, optionally in the ionized form, as stabilizing agent, wherein said colloids with apatite structure have the formula:
Ca₁₀₋ₓ(HPO₄)ₓ(PO₄)₆₋ₓ(J)₂₋ₓ (I)
in which:
x is selected from 0, 1 or 2;
J is selected from OH⁻, F⁻, CO₃²⁻ or Cl⁻;
and in which some phosphate ions (PO₄³⁻) or hydrogen-phosphate ions (HPO₄²⁻) can be replaced by carbonate ions (CO₃²⁻);
and in which some Ca²⁺ can be replaced by Mⁿ⁺ metal cations of alkali metals, alkaline earth metals or lanthanide metals where n represents 1, 2 or 3,
it being understood that the molar ratio of the Mⁿ⁺ cation, when it is present, to Ca²⁺ varies between 0.01:0.99 and 0.25:0.75, and that the substitution of HPO₄²⁻ ions or of PO₄³⁻ ions by CO₃²⁻ ions, the incorporation of CO₃²⁻ ions as J and the substitution of Ca²⁺ cations by metal cations is carried out so as to satisfy the electrostatic balance.

2. The colloidal dispersion as claimed in claim 1, characterized in that the molar ratio of the total Ca²⁺ to the total P in the colloidal phase varies between 1.3 and 1.7.

3. The colloidal dispersion as claimed in either one of claims 1 and 2, characterized in that the molar ratio of the total stabilizing agent to the total Ca²⁺ in the colloidal phase varies between 0.001 and 1.0.

4. The colloidal dispersion as claimed in any one of claims 1 to 3, characterized in that x represents 0.

5. The colloidal dispersion as claimed in any one of claims 1 to 4, in which the colloids possessing an apatite structure have the formula: Ca₁₀(PO₄)₆(OH)₂.

6. The colloidal dispersion as claimed in any one of claims 1 to 5, characterized in that the stabilizing agent is selected from lysine, glycine, asparagine, creatine, arginine, aspartic acid, glutamic acid, serine, alanine, valine, leucine, their salts with acids or bases, and their mixtures.

7. The colloidal dispersion as claimed in claim 6, characterized in that the stabilizing agent is selected from lysine, creatine, glycine, alanine, asparagine, serine, their salts with acids or bases, and their mixtures.

8. The colloidal dispersion as claimed in any one of claims 1 to 7, exhibiting a concentration of calcium in the form of colloids possessing an apatite structure of greater than 0.25M, advantageously of greater than 0.5M.

9. A process for the preparation of a stable aqueous colloidal dispersion comprising the stages consisting in:
a) bringing into contact, in aqueous solution, a source of Ca cations, a source of PO₄³⁻ anions and an amino acid or a salt of such an amino acid with an acid or a base as stabilizing agent, at a pH of between 5 and 10, the respective amounts of the source of Ca²⁺ and of the source of PO₄³⁻ anions being such that the Ca²⁺/P molar ratio varies between 1 and 3.5, preferably between 2 and 3.2, the amount of stabilizing agent being such that the stabilizing agent/Ca²⁺ molar ratio varies between 0.3 and 2.5, preferably between 0.9 and 2;
b) optionally leaving the solution thus obtained to mature at a temperature of between 15 and 150° C. until a colloidal dispersion is obtained.

10. The process as claimed in claim 9, characterized in that the temperature is maintained between 40 and 150° C. in stage b).

11. The process as claimed in either one of claims 9 and 10, characterized in that the solution obtained at the conclusion of stage b) is concentrated by ultrafiltration.

12. The process as claimed in any one of claims 9 to 11, characterized in that the stabilizing agent is as defined in either one of claims 7 and 8.

13. The process as claimed in any one of claims 9 to 12, characterized in that, in stage a), the source of Ca²⁺ and the source of PO₄³⁻ are brought into contact by mixing an aqueous solution of a source of PO₄³⁻ with an aqueous solution of a source of Ca²⁺ comprising the stabilizing agent.

14. The process as claimed in any one of claims 9 to 13, characterized in that the source of calcium is selected from calcium hydroxide, calcium oxides, calcium halides, calcium nitrate and calcium hydrogencarbonate.

15. The process as claimed in any one of claims 9 to 14, characterized in that the source of PO₄³⁻ is selected from the salts of PO₄³⁻, H₂PO₄⁻ or HPO₄²⁻ anions, such as the alkali metal salts and the ammonium salts.

16. The process as claimed in any one of claims 9 to 15, characterized in that the pH is adjusted to between 7 and 9.2 in stage a).

17. A water-redispersible colloid possessing an apatite structure which can be obtained by carrying out the stages consisting in:
a) preparing a colloidal dispersion by employing the process as claimed in any one of claims 9 to 16;
b) isolating the colloid from the colloidal dispersion resulting from stage a).

18. The process as claimed in claim 9 for the preparation of a transparent colloidal dispersion, characterized in that one or more of the following conditions a) to d) are fulfilled:
a) the molar ratio of the stabilizing agent to the calcium is greater than 0.5:1, better still greater than 1:1;
b) the pH is between 5 and 9.5, better still between 7 and 9.5;
c) the stabilizing agent is composed of one or more amino acids, optionally in the ionized form, and is selected from lysine, alanine and their ionized forms;
d) the source of PO₄³⁻, the source of Ca²⁺ and the stabilizing agent are brought into contact by addition of the source of PO₄³⁻ to the solution of the source of Ca²⁺, which comprises the stabilizing agent, or vice versa.

19. The process as claimed in claim 18, characterized in that the conditions a) to d) are fulfilled.

20. A colloidal dispersion which can be obtained according to the process of either one of claims 18 and 19.

21. The transparent colloidal dispersion as claimed in claim 1, characterized in that it is formed of colloids of oblong shape with a (number-)average length of 20 to 150 nm or of spherical shape with a diameter of 10 to 100 nm, in which dispersion at least 80% of the colloids are not aggregated, the molar ratio of the stabilizing agent to the total calcium present in the colloids or at the surface of the colloids is between 0.001 and 1, the pH of the colloidal dispersion being between 5 and 9.5.

22. The transparent colloidal dispersion as claimed in claim 21, characterized in that the stabilizing agent is selected from alanine and lysine, optionally in the ionized form, and a mixture of these compounds.
